(19) United States
(12) Patent Application Publication
(10) Pub. No.: US 2009/0112233 A1
(43) Pub. Date: Apr. 30, 2009

(54) Prosthesis Fixation Apparatus and Methods

(75) Inventor: Jia Hua Xiao, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/928,379

(22) Filed: Oct. 30, 2007

(51) Int. Cl. A61B 17/10; A61B 17/08

(52) U.S. Cl. 606/143; 606/142; 606/151

(57) ABSTRACT

Endovascular fastener delivery apparatus comprises a flexible elongated member having a proximal end and a distal end and being configured and adapted to be endolumenally advanced through human vasculature, a flexible pusher member slidably coupled to the elongated member, and a plurality of serially aligned clip carriers secured to the pusher member, each clip carrier comprising a spring element and having a leading end adapted to seat a fastener and a trailing end fixedly secured to the pusher member. In one embodiment, a plurality of bridge clip fasteners are endolumenally advanced to a site in a lumen in a human body and the fasteners are passed from an inner surface of the prosthesis through the prosthesis and a wall of the lumen to secure the prosthesis to the lumen wall.

### FIELD OF THE INVENTION

The invention relates to prosthesis fixation in a passageway in a human body such as an artery.

### BACKGROUND OF THE INVENTION

Tubular prostheses such as stents, grafts, and stent-grafts (e.g., stents having an inner and/or outer covering comprising graft material and which may be referred to as covered stents) have been used to treat abnormalities in passageways in the human body. In vascular applications, these devices often are used to replace or bypass occluded, diseased or damaged blood vessels such as stenotic or aneurysmal vessels. For example, it is well known to use stent-grafts, which comprise biocompatible graft material (e.g., Dacron® or expanded polytetrafluoroethylene (ePTFE)) supported by a framework (e.g., one or more stent or stent-like structures), to treat or isolate aneurysms. The framework provides mechanical support and the graft material or liner provides a blood barrier.

Aneurysms generally involve abnormal widening of a duct or canal such as a blood vessel and generally appear in the form of a sac formed by the abnormal dilation of the duct or vessel wall. The abnormally dilated wall typically is weakened and susceptible to rupture. Aneurysms can occur in blood vessels such as in the abdominal aorta where the aneurysm generally extends below the renal arteries distally to or toward the iliac arteries.

In treating an aneurysm with a stent-graft, the stent-graft typically is placed so that one end of the stent-graft is situated proximally or upstream of the diseased portion of the vessel and the other end of the stent-graft is situated distally or downstream of the diseased portion of the vessel. In this manner, the stent-graft extends through the aneurysmal sac and beyond the proximal and distal ends thereof to replace or bypass the weakened portion. The graft material typically forms a blood impervious lumen to facilitate endovascular exclusion of the aneurysm.

Such prostheses can be implanted in an open surgical procedure or with a minimally invasive endovascular approach. Minimally invasive endovascular stent-graft use is preferred by many physicians over traditional open surgery techniques where the diseased vessel is surgically opened and a graft is sutured into position such that it bypasses the aneurysm. The endovascular approach, which has been used to deliver stents, grafts, and stent grafts, generally involves cutting through the skin to access a lumen of the vasculature. Alternatively, lumenar or vascular access may be achieved percutaneously via successive dilation at a less traumatic entry point. Once access is achieved, the stent-graft can be routed through the vasculature to the target site. For example, a stent-graft delivery catheter loaded with a stent-graft can be percutaneously introduced into the vasculature (e.g., into a femoral artery) and the stent-graft delivered endovascularly across the aneurysm where it is deployed.

When using a balloon expandable stent-graft, balloon catheters generally are used to expand the stent-graft after it is positioned at the target site. When, however, a self-expanding stent-graft is used, the stent-graft generally is radially compressed or folded and placed at the distal end of a sheath or delivery catheter. Upon retraction or removal of the sheath or catheter at the target site, the stent-graft self-expands.

More specifically, a delivery catheter having coaxial inner and outer tubes arranged for relative axial movement therebetween can be used and loaded with a compressed self-expanding stent-graft. The stent-graft is positioned within the distal end of the outer tube (sheath) and in front of a stop fixed to the inner tube. Once the catheter is positioned for deployment of the stent-graft at the target site, the inner tube is held stationary and the outer tube (sheath) withdrawn so that the stent-graft is gradually exposed and allowed to expand. The inner tube or plunger prevents the stent-graft from moving back as the outer tube or sheath is withdrawn. An exemplary stent-graft delivery system is described in U.S. Patent Application Publication No. 2004/0093063, which published on May 13, 2004 to Wright et al. and is entitled Controlled Deployment Delivery System, the disclosure of which is hereby incorporated herein in its entirety by reference.

Regarding proximal and distal positions referenced herein, the proximal end of a prosthesis (e.g., stent-graft) is the end closer to the heart (by way of blood flow) whereas the distal end is the end farther away from the heart during deployment. In contrast, the distal end of a catheter is usually identified as the end that is farthest from the operator, while the proximal end of the catheter is the end nearest the operator.

Although the endolumenal approach is much less invasive, and usually requires less recovery time and involves less risk of complication as compared to open surgery, among the challenges with this approach are fixation of the prosthesis (migration of prosthesis) and sealing of the prosthesis. For example, the outward spring force of a self-expanding stent-graft may not be sufficient to prevent migration. This problem can be exacerbated when the vessel's fixation zone significantly deviates from being circular. And when there is a short landing zone, for example, between an aortic aneurysm and a proximal branching artery (e.g., one of the renal arteries, or the carotid or brachiocephalic artery), small deviations in sizing or placement may result in migration and or leakage.

Current endovascular devices incorporate stent-graft over-sizing to generate radial force for fixation and/or sealing and some have included fixation mechanisms comprising radially extending members such as tines, barbs, hooks and the like that engage the vessel wall to reduce the chance of migration. In some abdominal aortic aneurysm applications, a suprarenal stent and hooks are used to anchor the stent-grafts to the aorta. However, abdominal aortic aneurysm stent-grafts typically require an anchor or landing zone of about 10-15 mm to achieve the desired fixation and seal efficacy. In some cases, such an anchoring or landing zone does not exist due to diseased vasculature or challenging anatomy. One staple approach described in copending, co-owned U.S. Patent Application Publication 2007/0219627 by Jack Chu et al, which was filed on Mar. 17, 2006 and is entitled Prosthesis Fixation Apparatus and Methods, involves delivering a fastener having a proximal piercing end portion and a distal piercing end portion to a site where a prosthesis having a tubular wall has been placed in the passageway, which has a wall, advancing the proximal piercing end portion beyond the prosthesis, penetrating the proximal piercing end portion into the wall of the passageway without passing the proximal piercing end portion through the tubular wall of the prosthesis, and passing the distal piercing end portion through the tubular wall of the prosthesis and into the wall of the passageway. Other approaches to improve fixation and/or sealing between the prosthesis and an endolumenal wall have included using adhesives and growth factor (see e.g., copending, co-owned U.S. Patent Application Publication 2007/0233227 by Trevor Greenan, which was filed on Mar. 30, 2006 and is entitled Prosthesis with Coupling Zone and Methods. Another fixation approach described in copending, co-owned U.S. patent application Ser. No. 11/736,453 by Jia Hua Xaio et al., filed Apr. 17, 2007 and entitled Prosthesis Fixation Apparatus and Methods, involves endolumenally advancing fasteners to a plurality of sites within a prosthesis such as a stent-graft and passing the fasteners from an inner surface of the prosthesis through the prosthesis and a wall of the passageway to which the prosthesis is to be secured. In one embodiment, the fasteners are deployed simultaneously and in another embodiment they are deployed serially.

There remains a need to develop and/or improve seal and/or fixation approaches for endolumenal or endovascular prosthesis placement.

### SUMMARY OF THE INVENTION

The present invention involves improvements in prosthesis fixation. In one embodiment according to the invention, a method of securing a tubular prosthesis to an inner wall of a vessel in a human patient comprises endolumenally advancing a bridge clip delivery device, which has a plurality of bridge clips, through a vessel in a patient and into a prosthesis that is in the vessel, each clip having a central portion and two end portions, each end portion having a memory shaped loop configuration and a piercing end, where the central portion forms a bridge that connects the two end portions and spaces them from one another; and deploying the clips from the delivery device with the piercing ends of a respective clip leading the clip while passing the piercing ends from the inner surface of a prosthesis, through the prosthesis and the vessel to secure the prosthesis to the vessel.

In another embodiment according to the invention, endovascular fastener delivery apparatus comprises a flexible elongated member having a proximal end and a distal end and being configured and adapted to be endolumenally advanced through human vasculature; a flexible pusher member slidably coupled to the elongated member; and a plurality of serially aligned clip carriers secured to the pusher member, each clip carrier comprising a spring element and having a leading end adapted to seat a fastener and a trailing end fixedly secured to the pusher member.

In another embodiment according to the invention, endovascular fastener delivery apparatus comprises a flexible elongated member having a proximal end and a distal end and adapted to be endolumenally advanced through human vasculature; a flexible pusher member slidably coupled to the elongated element; and a plurality of serially aligned clip carriers secured to the pusher element, each clip carrier comprising a spring element and having a leading end adapted to seat a fastener and a trailing end secured to the pusher element; a plurality of bridge clips, each clip having a central portion seated in one of the carriers, and two end portions, each end portion having a memory shaped loop configuration and a piercing end, where the central portion forms a bridge that connects the two end portions and space them from one another.

The above is a brief description of some deficiencies in the prior art and advantages of embodiments according to the present invention. Other features, advantages, and embodiments according to the present invention will be apparent to those skilled in the art from the following description and accompanying drawings, wherein, for purposes of illustration only, specific embodiments are set forth in detail.

### BRIEF DESCRIPTION OF THE DRAWINGS

FIG. 1 diagrammatically illustrates an endovascular fastener delivery apparatus according to one embodiment of the invention.

FIG. 2A depicts a bridge clip for use in the delivery apparatus of FIG. 1 in a restrained open configuration.

FIG. 2B depicts the clip of FIG. 2A in an unrestrained memory set closed configuration.

FIG. 3 is an exploded view of a distal end portion of the apparatus of FIG. 1.

FIG. 4 is a partial sectional view of a distal end portion of the apparatus of FIG. 1.

FIG. 5 is a sectional view of the delivery apparatus of FIG. 4 taken along line 5-5.

FIG. 6 illustrates a first state of deployment of a clip from the apparatus of FIG. 4.

FIG. 7 illustrates further deployment of the clip of FIG. 6.

FIG. 8 illustrates retraction of the clip pusher of the clip delivery apparatus deployment of the clip of FIG. 6.

FIG. 9 illustrates the clip pusher re-engaged in a subsequent set of clips and ready to deploy a second clip.

FIGS. 10A-G illustrate securing a prosthesis in the region of an aortic abdominal aneurysm where FIG. 10A illustrates endolumenally delivering a prosthesis such as a stent-graft to a target site; FIG. 10B illustrates the prosthesis of FIG. 10B deployed with one end portion positioned along a landing zone between a branch vessel and an aneurysm; FIG. 10C is a sectional view of a portion of the vessel of FIG. 10B illustrating an end view of the distal end of the fastener delivery apparatus of FIG. 1 after it has been endolumenally delivered and positioned along the landing zone and secured in place with a balloon as a first clip is deployed through the stent-graft and aorta to secure the stent-graft to the aorta; FIG. 10D shows the first clip in place and the fastener delivery apparatus balloon deflated and the apparatus rotated to prepare the apparatus for deploying a second clip at a second location; FIG. 10D1 shows a view taken along line 10D1-10D1 of the clip of FIG. 10D; FIG. 10E illustrates the balloon inflated to secure the distal end of the fastener delivery apparatus at a second location and deployment of a second clip about 180 degrees from the first clip; FIG. 10F depicts a securement configuration where six clips secure the prosthesis to the landing zone or proximal landing of the abdominal aortic aneurysm; and FIG. 10F is a partial sectional view of the prosthesis secured to the vessel with the fasteners and with its contralateral leg attached.

### DETAILED DESCRIPTION

The following description will be made with reference to the drawings where when referring to the various figures, it should be understood that like numerals or characters indicate like elements. Further, when referring to catheters, delivery devices, and loaded fasteners described below, the proximal end is the end nearest the operator and the distal end is farthest from the operator.

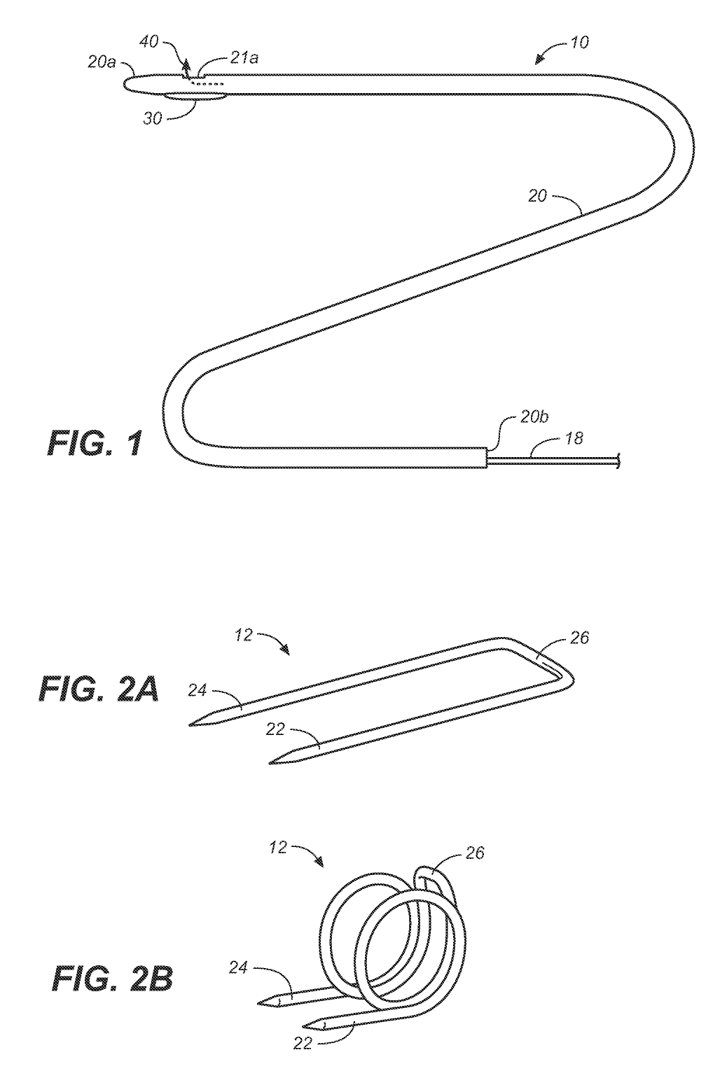

Referring to FIG. 1, one embodiment of endolumenal fastener delivery apparatus according to the invention is shown and generally designated with reference numeral 10. Endolumenal fastener delivery apparatus 10 comprises an elongated member 20 and a fastener push or pusher member 18 slidably mounted in a channel in elongated member 20. Elongated member 20 and push member 18 are sufficiently flexible and of suitable length to be endolumenally delivered to a desired site in a human patient. In one example, they are sufficiently flexible to be introduced through the femoral artery to the aorta where distal end 20a of elongated member 20 is positioned beyond an abdominal aortic aneurysm to deploy a fastener and secure a prosthesis, which has been positioned to bypass the aneurysm, to the aorta as will be described in more detail below. Push member 18 and elongated member 20 can comprise any suitable material and in one example can comprise PEEK, which is an abbreviation for PolyEtherEtherKetones. The length of elongated member 20 measured from its distal end 20a to its proximal end 20b typically ranges from about 50 cm to about 110 cm and depends on the application. When used in connection with securing grafts or stent-grafts to the inner wall of the aorta to bypass an abdominal aortic aneurysm, its length typically will be about 50 cm to about 70 cm and in thoracic applications where it is used to endolumenally secure grafts or stent-grafts to the inner wall of a vessel, its length typically will be about 70-110 cm. The length of push member 18 typically will be greater than that of elongated member 20 so that there is a sufficient length at it its proximal end for the operator to grip and manipulate.

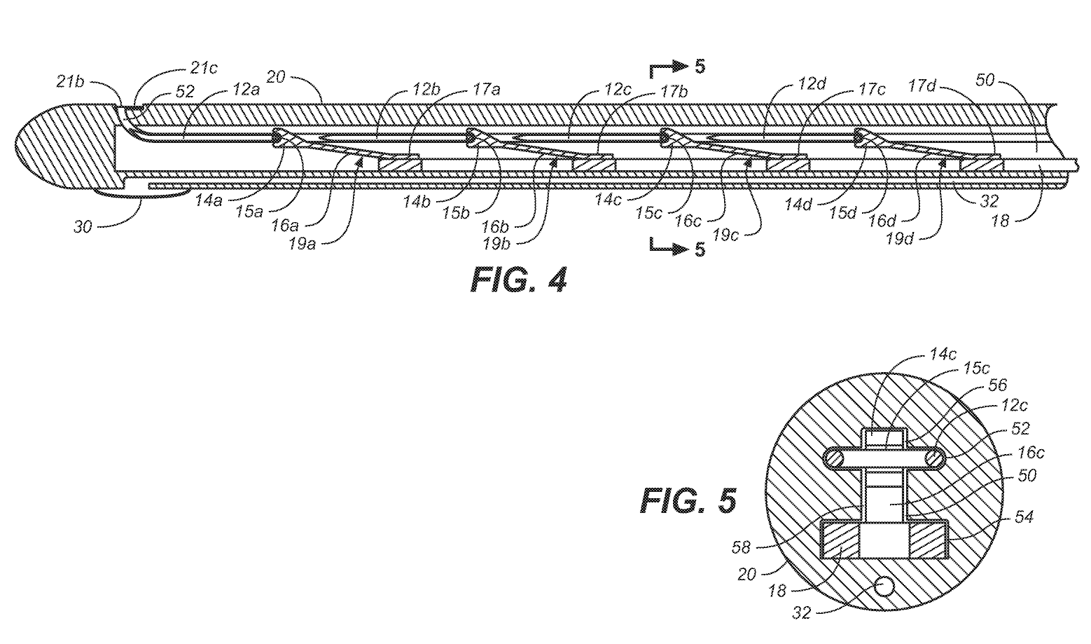

Endolumenal fastener delivery apparatus 10 further comprises a plurality of carriers adapted to carry fasteners. In the illustrative embodiment, the carriers are fixedly secured to push member 18. When delivery apparatus 10 is loaded with fasteners, push member 18 can be advanced to eject a fastener from a side of elongated member 20 as diagrammatically shown with arrow 40 in the embodiment illustrated in FIG. 1. Elongated member 20 can be provided with a recess 21a having an opening 21b to which fastener channel 52 (see FIGS. 4 & 5) extends. After a fastener is deployed, push member 18 is retracted to associate the lead carrier with another fastener so that another fastener can be deployed as will be described in more detail below. Fastener delivery apparatus 10 also can include an expansion device to fix the position of distal end 20a during endolumenal fastener deployment. In the embodiment illustrated in FIG. 1, such an expansion device is in the form of expandable balloon 30. It should be understood, however, that other expansion mechanisms can be used. Further, although elongated member 20 is shown with a side ejection opening, the opening can positioned at the end of the member.

One embodiment of a fastener that can be used with fastener delivery system 10 is shown in FIGS. 2A and 2B and generally designated with reference numeral 12. FIG. 2A depicts fastener 12, which is in the form of a bridge clip, in a restrained open configuration and FIG. 2B depicts the fastener 12 in a closed configuration. Fastener or bridge clip 12 comprises a first leg portion 22, a second leg portion 24, and a central or bridge portion 26, which connects or bridges leg portions 22 and 24 and maintains the legs spaced from one another. Each leg portion has a sharp piercing end. Bridge portion 26 is substantially straight and has a length that typically is about 2 to 5 mm.

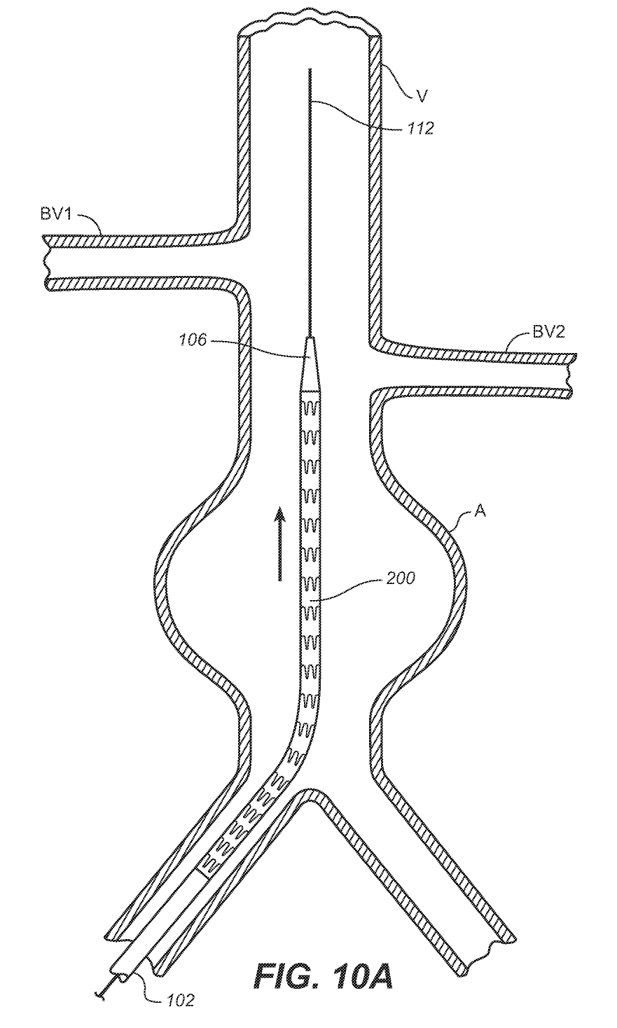

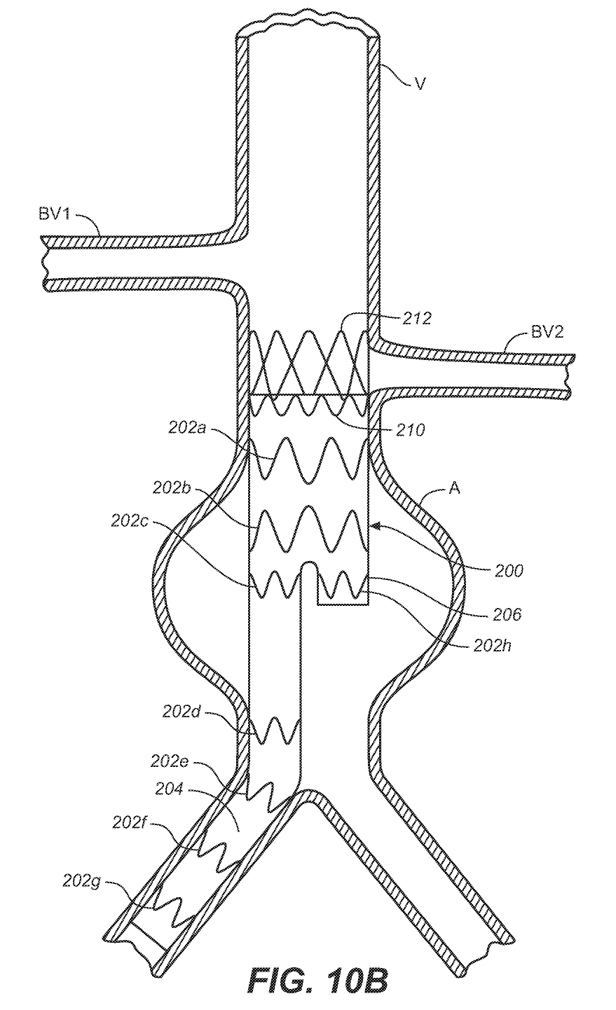

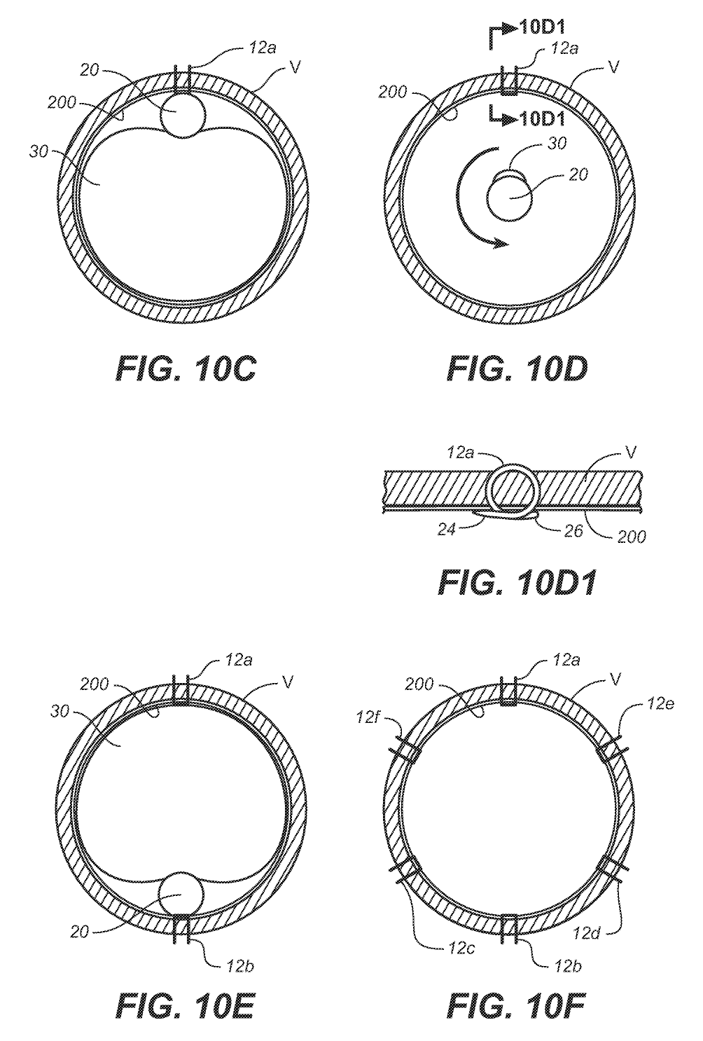

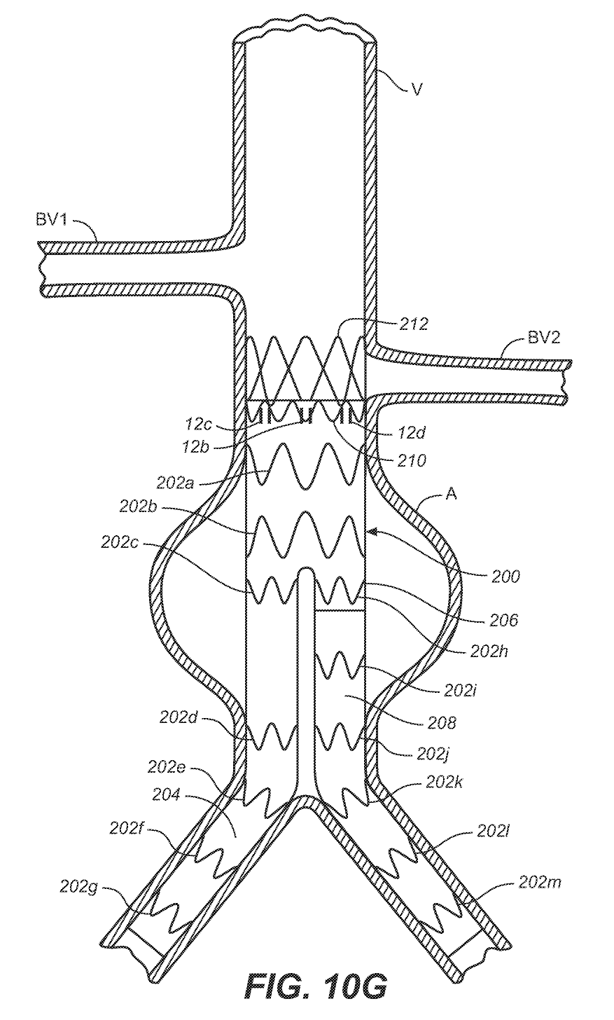

Referring to FIG. 2A, leg portions 22 and 24 are shown restrained or deformed so as to provide a clip having a generally planar configuration that is suitable for delivery through channel 52 (FIG. 5) of elongated member 20. In FIG. 2B, clip 12 is shown in a free state where leg portions 22 and 24 have returned to a loop shaped memory set configuration. The lengths of the straight end portions are typically the same and typically are dimensioned to be slightly greater than the thickness of the vessel wall and graft material to be pierced so that the they extend beyond the outer surface of the vessel wall before looping back through the vessel wall as the clip moves toward its memory set configuration (see e.g., FIG.10D1). In this manner, a segment of the clip can be positioned completely outside the outer surface of the vessel wall with an outer surface of the segment facing an outer surface of the vessel wall to optimize the attachment therebetween. Clip 12 can comprise any suitable material. It can be made from shape memory material such as nitinol wire. When using nitinol wire, clip 12 can be placed in the desired shape (e.g., that shown in FIG. 2B) and heated for about 5-15 minutes in a hot salt bath or sand having a temperature of about 480-515° C. The clip can then be air cooled or placed in an oil bath or water quenched depending on the desired properties. In one alternative, the fasteners can be surgical grade stainless steel that is deformed to assume such a preshaped configuration. In a further embodiment, the fasteners can be polymeric material with a preshaped loop configuration to which they return when released from the fastener tube.

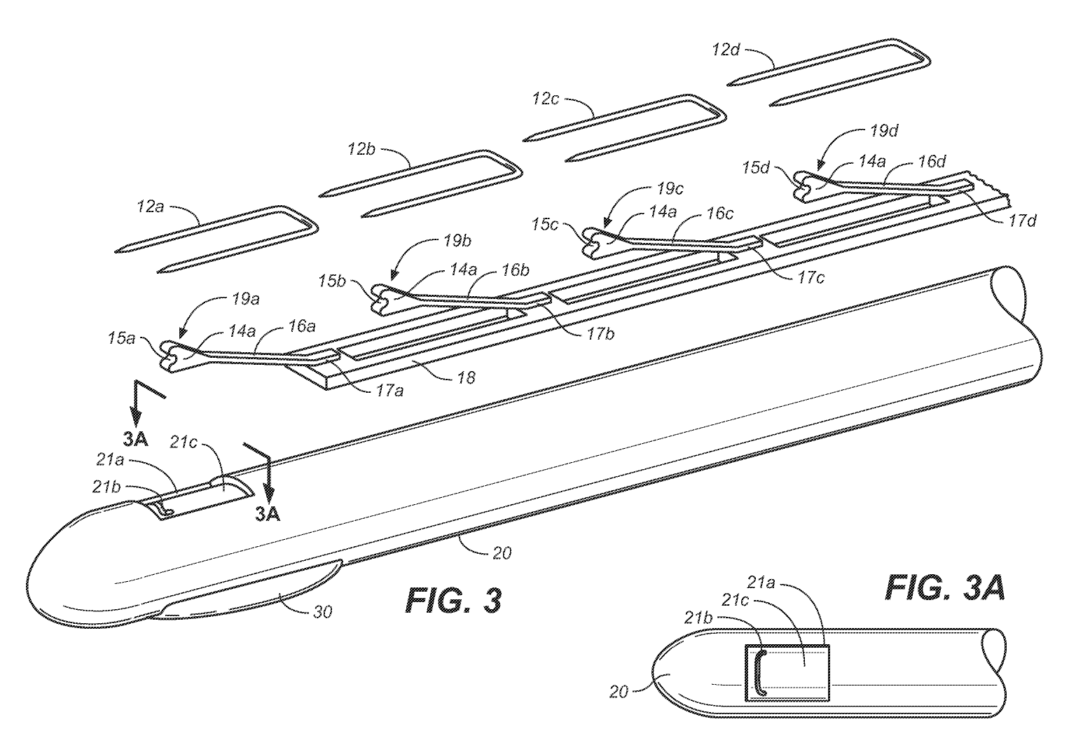

Referring to FIG. 3, an exploded view of a distal portion of fastener delivery apparatus 10 illustrates clips 12a,b,c,d and push member 18 with clip carriers 19a,b,c,d separated from and outside elongated member 20. Although four clips and clip carriers are shown, the number of clips and/or clip carriers can vary depending on the application. When securing a bifurcated graft or stent-graft to an aorta to bypass an abdominal aortic aneurysm, the number of clips used typically will be about two to six, and more typically, three to four clips. In general, at least two clips are used.

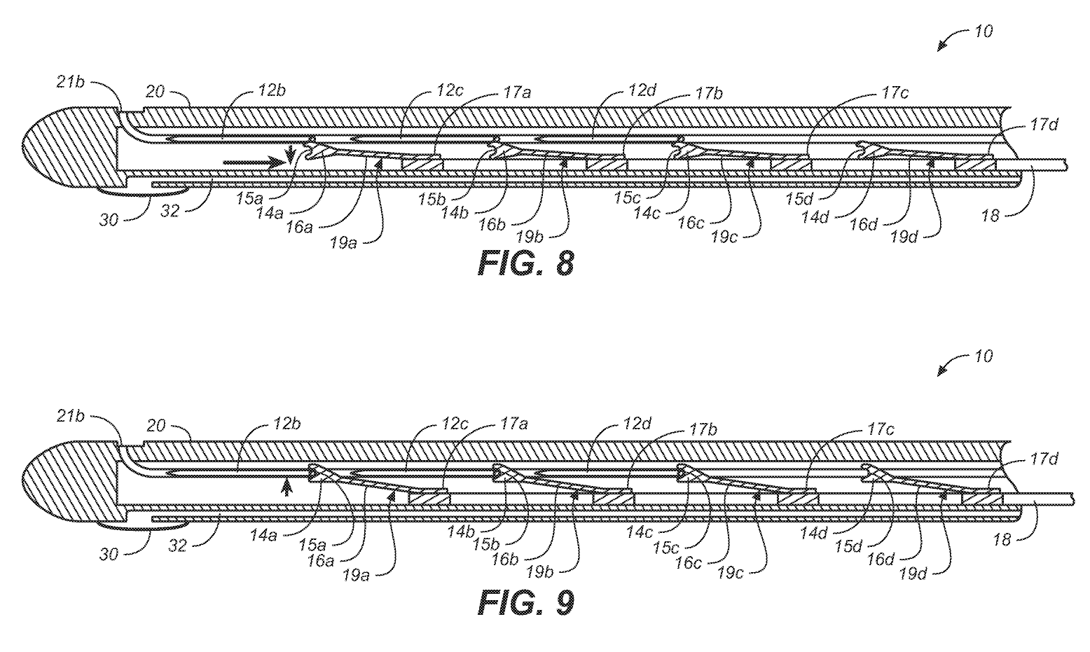

Clip carriers 19a,b,c,d . . . n include one end 17a,b,c,d . . . n secured to push member 18 (e.g., with glue or any conventional fusion or bonding technique), an enlarged end 14a,b,c,d . . . n with a recess 15a,b,c,d . . . n adapted to receive a respective bridge portion 26 of one of clips 12a,b,c,d . . . n, and a general flat central section 16a,b,c,d . . . n. The clip carrier material can be selected from any suitable material to provide the spring characteristics depicted in FIGS. 8 and 9 and described below. The clip carriers can, for example, be made from nitinol, stainless steel, or spring steel. In the illustrative embodiment, push member 18 has a plurality of clip carrier receiving portions in the form of rectangular shaped through holes that are aligned with carriers 19a,b,c,d . . . n. Although three such receiving portions or holes are shown, they can be provided along the entire length of push member 18. The receiving portions or through holes are made to allow the clip carrier 19a,b,c,d . . . n to bend beyond the surface of push member 18 so that, for example, at least a portion of enlarged end portions 14a,b,c,d . . . n can be pushed and received therein. In this manner, the distance between push member 18 and clip channel 52 can be reduced to minimize the diameter or profile of elongated member 20. Accordingly, there are at least as many receiving portions or through holes as there are clip carriers in this embodiment. It should be understood, however, that other receiving portion or through hole configurations and patterns can be used. For example, the through holes can be replaced with recesses that provide sufficient space for receiving at least a portion of the clip carriers. Further, the through holes are optional and push member 18 can be provided without through holes.

In use, push member 18 is advanced in channel 50, which is formed or provided in elongated member 20, to eject fasteners through opening 21b, which also is formed in elongated member 20. Elongated member 20 can be an extruded to form channel 50. In the illustrative embodiment, opening 21b is formed in the surface of elongated member recess 21a, which provides a surface on which a radiopaque marker 21c can be placed. Clip channel 52 extends to opening 21b to facilitate the ejection of the fasteners. If desired, the portion of channel 50 through which carrier head or enlarged portion 14 passes also can be extended to opening 21b. However, when only the portion of channel 50 that corresponds to clip channel portion 52 extends to opening 21b, the inner surface of the elongated member facing or forming part of the channel provides a stop to preclude the carrier from extending out from the elongated member and contacting vasculature. In another embodiment, such a recess is not provided and channel 52 or a larger channel as described above to accommodate carrier head or enlarged portion 14 is extended to the outer surface of elongated member 20. In a similar manner, a radiopaque marker can be provided adjacent to or around the opening where the channel opens into the outer surface of the elongated member. In this arrangement, the recess is optional.

When a radiopaque plate or surface is provided so as to surround opening 21b as shown in FIG. 3, the void in the radiopaque material where the opening is positioned can provide an indication of the direction and/or orientation of opening 21b based on the known relative position and orientation of the opening and radiopaque marker. Conventional fluoroscopic imaging techniques can be used to provide an image of the direction and/or orientation of the opening. This can assist the physician in positioning the opening at a target site where a fastener is to be deployed. Opening 21b can be straight or curved. A curved shaped opening may enhance opening direction and orientation detection when surrounded and bordered by radiopaque material. Opening 21b can have a “[” bracket shape, a “C” shape, a crescent shape or any other suitable shape for providing a port for the clips to exit and a shape that provides the desired fluoroscopic detection when surrounded and bordered by radiopaque material. In a further alternative the radiopaque material can be provided adjacent to the opening and when the dimensions of distal end 20, opening 21b and the marker are known, fluoroscopic imaging can indicate the direction, orientation and position of the opening.

In another alternative, a radiopaque marker having an “e” shape can be placed adjacent to the opening (e.g., opening 21b) through which the fastener is ejected. For example, a platinum wire having and “e” shape and positioned in a known orientation relative to opening 21b and distal end 20a of elongated member 20 can provide an indication of the direction and orientation of distal end 20a and/or opening 21b based on known positions and orientations of these elements relative to one another.

Referring to FIG. 4, a partial sectional view of a distal portion of fastener delivery apparatus 10 is shown and illustrates the position of clips 12a-d, carriers 19a-d, and push member 18 in channel 50 of elongated member 20, which, as noted above, can be formed as an extrusion. Fluid pressure lumen 32 also can be formed in a wall of elongated member 20 to extend from the proximal end 20b of elongated member 20 to an outer surface of member 20 below balloon 30 to provide a means to inflate or deflate balloon 30.

Bridge portions 26 of bridge clips 12 are seated in recesses 15a,b,c,d, which are formed in the enlarged leading portions 14a,b,c,d of carriers 19a,b,c,d. Carrier attachment portions 17a,b,c,d are secured to push member 18 with spring beam portions 16a,b,c,d extending out of the plane of the attachment surface of push member 18 in a direction toward channel 52 in which the clips are slidably positioned. The spring beam portions 16a,b,c,d are biased or shaped to be in the position depicted in FIG. 3, but they and enlarged portions 14a,b,c,d can be deflected toward the push member when retracted and passed under clip bridge portions 26 as will be described in more detail below. Although four clips and clip carriers are shown for purposes of example, more or fewer clips and clip carriers can be used.

Referring to FIG. 5, one channel configuration is shown according to one embodiment of the invention. In this embodiment, channel 50 includes clip channel portion 52, which is configured to accommodate and provide a guide for the clips, push member channel portion 54, which is configured to accommodate and provide a guide for push member 18, upper channel portion 56, which is configured to accommodate and provide a guide for the upper portions of carrier portions 14a,b . . . n, and intermediate or central channel portion 58, which is configured to accommodate and provide a guide for the lower portions of carrier portions 14a,b . . . n and central spring beam portions 16a,b . . . n. In general, the illustrative embodiment shows one suitable configuration where channel 50 has the same configuration as a transverse section of the pusher member, carrier and clip throughout the length of those elements. Although channel 50 forms a track for the fasteners, fastener carriers, and push member in the embodiment described above, other track arrangements can be used. For example, a track for the fasteners, fastener carriers, and push member can be provided on the exterior of elongated member 20 or it can be provided in a recess formed therein.

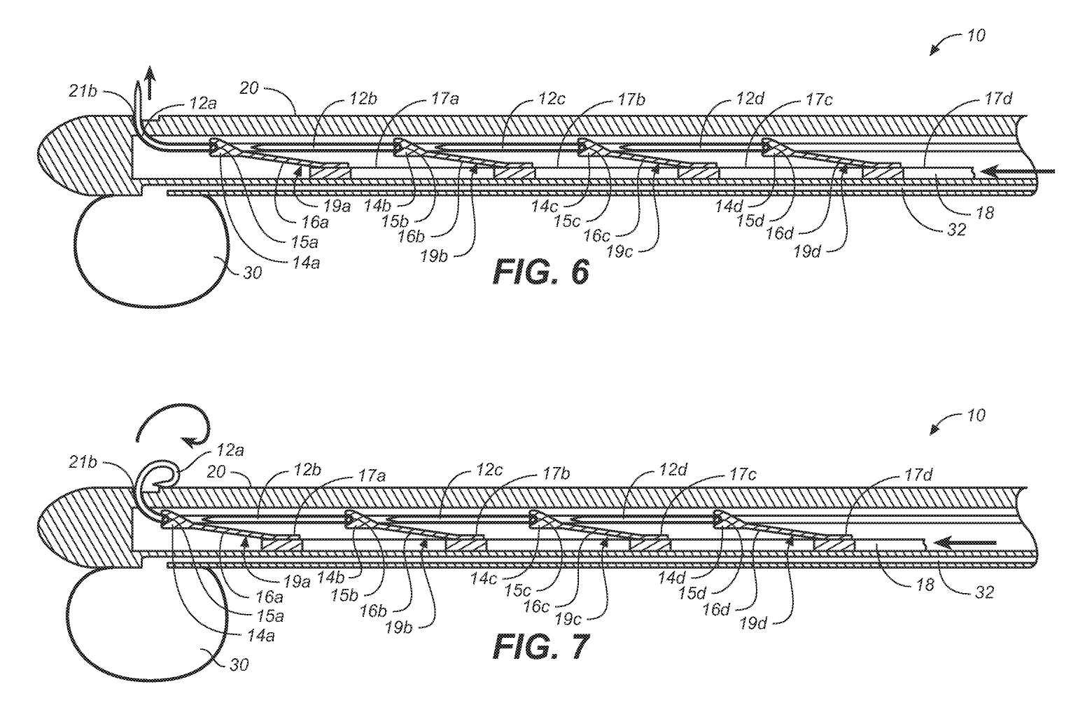

Referring to FIGS. 6-9, clip deployment and reloading are shown. Referring first to FIG. 6, after elongated member 20 is at the desired position in a prosthesis, balloon 30 is inflated so that the balloon contacts the inner wall of the surrounding prosthesis and urges the distal end of elongated member 20 against an inner wall portion of the surrounding prosthesis (e.g., the inner wall surface opposite the balloon) to fix the position of clip deployment opening 21b. Then push member 18 is moved distally to move all of the carriers 19a,b . . . n distally at the same time with lead carrier 19a pushing lead clip 12a out through opening 21b. As push member 18 is further advanced distally, lead clip 12a is further ejected as shown in FIG. 7. After lead clip 12a is completely ejected, balloon 30 can be deflated and push member 18 retracted (FIG. 8). As push member 18 is retracted, bridge clip bridge portions 26 deflect each carrier member to slide beneath the bridge portions as shown in FIG. 8. When the leading portions 14a,b . . . n pass beneath bridge portions 26, they move or spring back into channel 52 to engage the remaining clips as shown in FIG. 9 where the clips again are seated in the carrier recesses. Clip delivery apparatus 10 is then ready to deploy clip 12b. These steps are continued until the desired number of clips has been deployed. In the illustrative embodiment, the wall surfaces that form channel 52 form a restraint that restrains the bridge clips in a generally planar configuration as shown, for example, in FIG. 2A. Since the bridge clips tend to move toward their closed configuration, they press against these wall surfaces. The friction between these elements or resultant resistance to clip movement minimizes or eliminates clip movement as the clip carriers move under the clips.

Referring to FIGS. 10A-G, an example method of using fastener delivery apparatus 10 will be described. In this example, a bifurcated stent-graft is used to bypass an abdominal aortic aneurysm “A.” A sheath is inserted through the femoral artery as is known in the art to provide access to a femoral artery, while maintaining hemostasis. Using conventional techniques, the stent-graft is percutaneously delivered through the sheath to the vicinity of the abdominal aortic aneurysm “A” where it is deployed. The stent-graft delivery catheter removed is removed and fastener delivery apparatus 10 introduced to fixedly secure the stent-graft to vessel.

FIG. 10A illustrates endolumenally delivering the distal end of a stent-graft delivery catheter 102 containing a self-expanding stent-graft 200 and having a flexible tapered tip 106 at its distal end to a target site (i.e., the proximal landing zone of an abdominal aortic aneurysm). Referring to FIG. 10B, once the catheter is in the desired position, the stent-graft is deployed and allowed to expand against vessel “V” (which in this example is the aorta) in the region below branch vessels BV1 and BV2 (which correspond to the renal arteries) as shown in FIG. 10B. One example of a suitable stent-graft deployment system is described in U.S. Patent Application Publication No. 2004/0093063, which published on May 13, 2004 to Wright et al. and is entitled Controlled Deployment Delivery System.

Once the stent-graft has been deployed and the stent-graft delivery catheter removed, fastener delivery apparatus 10 is introduced through the femoral artery and advanced into the stent-graft lumen where its distal end is positioned adjacent to or in the vicinity of the proximal landing zone of aneurysm “A.” Elongated member 20 can be introduced through the same sheath that provided access for the stent-graft delivery catheter. The radiopaque marker facilitates positioning the distal end of elongated member 20 using conventional fluoroscopic techniques.

Referring to FIG. 10C, once the distal end of elongated member 20 or clip ejection opening 21b is at the desired position, balloon 30 is inflated to urge elongated member 20 against the inner wall surface of stent-graft 200 in vessel “V.” This fixes the position of a distal end portion of elongated member 20. Push member 18 is then advanced to eject clip 12a. After the clip is ejected, balloon 30 is deflated and elongated member 20 rotated about 180 degrees as shown in FIG. 10D. FIG. 10D1 shows a side view of the clip of FIG. 10D rotated 90 degrees as compared to that shown in FIG. 10D. Referring to FIG. 10E, the balloon is expanded again and clip 12b ejected at a position about 180 degrees from clip 12a after which the balloon is deflated and rotated for deploying another clip. After the desired number of clips has been deployed, fastener delivery apparatus is withdrawn and contralateral leg portion 208, which can include a tubular graft member and annular wire springs or stents 202i-m, is then secured to the graft member short leg portion 206 as is known in the art (FIG. 10G). Referring to FIG. 10F, one securement configuration where six clips 12a,b,c,d,e,f have been deployed to secure stent-graft 200 to the proximal landing zone of the abdominal aortic aneurysm is shown. It should be understood, however, that other clip configurations and numbers of clips can be used.

Returning to FIG. 10G, the fully deployed stent-graft includes ipsilateral leg 204 and contralateral stump 206 to which contralateral leg 208 is coupled. The combined prosthesis includes stent elements 202a-m. The stent elements provide structural support to the tubular graft elements as is known in the art. As shown in FIG. 10G, an undulating bare spring element 212 also can be sutured or otherwise attached to the proximal end of the prosthesis and/or an annular undulating wire 210 having an undulating configuration secured to the proximal end of the prosthesis to provide radial strength as well. The spring has a radially outward bias so that when it is released from a radially collapsed or restrained state it expands outwardly to secure the proximal portion of the prosthesis to the target passageway wall. Another undulating wire 210 can be attached to the prosthesis distal end as well or in the alternative. More specifically, a support spring 210 can be provided at one or both ends of the prosthesis. The stent and support elements can be positioned on the interior and/or exterior of the graft member and secured thereto by suturing or other conventional means. The stent framework can be nitinol or any other suitable material. The graft material also can be any suitable material such as Dacron®, PEEK, UHMWPE or expanded polytetrafluoroethylene (ePTFE). In a further embodiment, a graft by itself without a stent framework can be used.

Any feature described in any one embodiment described herein can be combined with any other feature of any of the other embodiments or features described herein. Furthermore, variations and modifications of the devices and methods disclosed herein will be readily apparent to persons skilled in the art.

What is claimed is:

1. A method of securing a tubular prosthesis to an inner wall of a vessel in a human patient comprising:
endolumenally advancing a bridge clip delivery device, which has a plurality of bridge clips, through a vessel in a patient and into a prosthesis that has been deployed in the vessel, each clip having a central portion and two end portions, each end portion having a memory shaped loop configuration and a piercing end, where the central portion forms a bridge that connects the two end portions and spaces them from one another; and
deploying the clips from the delivery device with the piercing ends of a respective clip leading the clip while passing the piercing ends from the inner surface of a prosthesis, through the prosthesis and the vessel to secure the prosthesis to the vessel.

2. The method of claim 1 wherein each fastener is seated in a movable carrier.

3. The method of claim 2 wherein the carriers are serially aligned and the carriers are moved to serially deploy the clips.

4. The method of claim 1 including using a fixation device to secure the position of the delivery device during clip deployment.

5. The method of claim 4 wherein the fixation device is an expandable element that is expanded to urge a portion of the delivery device against the inner surface of the prosthesis.

6. The method of claim 5 wherein the fixation device is a balloon.

7. The method of claim 5 wherein the fixation device is returned toward a non-expanded state after deployment of a one of said clips, a portion of the bridge clip delivery device moved to another location on the inner surface of the prosthesis, the fixation device expanded to secure to position of the bridge clip delivery device, and a another clip deployed to secure the prosthesis to the vessel.

8. The method of claim 1 wherein the prosthesis comprises a bifurcated stent-graft and the clips are passed through the vessel in a region between a branch vessel that branches from the vessel and an aneurysm.

9. The method of claim 8 wherein the vessel is the aorta and the clips are deployed to secure a portion of the bifurcated stent-graft to the proximal landing of an abdominal aortic aneurysm.

10. Endovascular fastener delivery apparatus comprising:
a flexible elongated member having a proximal end and a distal end and being configured and adapted to be endolumenally advanced through human vasculature;
a flexible pusher member slidably coupled to said elongated member; and
a plurality of serially aligned clip carriers secured to said pusher member, each clip carrier comprising a spring element and having a leading end adapted to seat a fastener and a trailing end fixedly secured to said pusher member.

11. The apparatus of claim 10 wherein said flexible elongated member has a length of at least about 50 cm.

12. The apparatus of claim 10 wherein said flexible elongated member has a length of about 50-110 cm.

13. The apparatus of claim 10 wherein said flexible elongated member has a channel in which said pusher member and carriers are slidably mounted.

14. The apparatus of claim 13 wherein said elongated member has an opening coupled to said channel for deployment of fasteners.

15. The apparatus of claim 14 wherein further including an expandable member in the vicinity of said opening.

16. The apparatus of claim 15 wherein said expandable member is a balloon.

17. The apparatus of claim 14 further including radiopaque marker material adjacent to said opening.

18. Endovascular fastener delivery apparatus comprising:
a flexible elongated member having a proximal end and a distal end and adapted to be endolumenally advanced through human vasculature;
a flexible pusher member slidably coupled to said elongated element; and
a plurality of serially aligned clip carriers secured to said pusher element, each clip carrier comprising a spring element and having a leading end adapted to seat a fastener and a trailing end secured to said pusher element;
a plurality of bridge clips, each clip having a central portion seated in one of said carriers, and two end portions, each end portion having a memory shaped loop configuration and a piercing end, where the central portion forms a bridge that connects the two end portions and spaces them from one another.

19. The apparatus of claim 18 wherein said flexible elongated member has a length of at least about 50 cm.

20. The apparatus of claim 18 wherein said flexible pusher member has a plurality of receiving portions formed therein, said receiving portions being aligned with said clip carriers such that at least a portion of each clip carrier can be received in one of said receiving portions.

21. The apparatus of claim 18 wherein said flexible elongated member has a channel in which said pusher member and carriers are slidably mounted.

22. The apparatus of claim 21 wherein said elongated member has an opening and said channel has a portion that extends to said opening for deployment of said clips.

23. The apparatus of claim 22 further including an expandable member coupled to said elongated member and circumferentially spaced from said opening.

24. The apparatus of claim 23 wherein said expandable member is a balloon.

25. The apparatus of claim 21 further including radiopaque marker material adjacent to said opening.
